(19) United States
(12) Patent Application Publication
(10) Pub. No.: US 2002/0042541 A1
(43) Pub. Date: Apr. 11, 2002

(54) Process for producing bis( trifluoromethyl)benzaldehyde

(76) Inventor: Takeo Komata, Saitama (JP)

Correspondence Address: CROWELL & MORING LLP, INTELLECTUAL PROPERTY GROUP, P.O. BOX 14300, WASHINGTON, DC, 20044-4300, US

(21) Appl. No.: 09/888,624

(22) Filed: Jun. 26, 2001

(30) Foreign Application Priority Data
Jun. 27, 2000 (JP) 2000-193314

(51) Int. Cl. C07C 45/49

(52) U.S. Cl. 568/429

(57) ABSTRACT

The invention relates to a process for producing a bis(trifluoromethyl)benzaldehyde represented by the general formula [1]. This process includes reacting a mono-substituted bis(trifluoromethyl)benzene, represented by the general formula [2], with carbon monoxide and hydrogen in the presence of a catalyst and a base, the catalyst including a palladium compound and a phosphine,

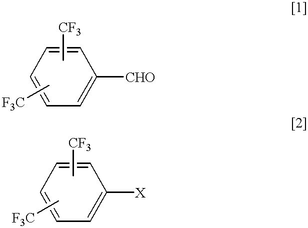

wherein X is a halogen atom selected from F, Cl, Br and I, a trifluoromethanesulfonate group, or a pentafluoroethanesulfonate group.

### BACKGROUND OF THE INVENTION

The present invention relates to processes for producing bis(trifluoromethyl)benzaldehyde represented by the general formula [1].

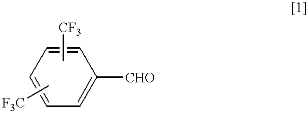

Bis(trifluoromethyl)benzaldehyde, represented by the general formula [1], is a useful compound as an intermediate for producing medicines, agricultural chemicals and various functional materials.

Eriks, J. C. et al., Eur. J. Med. Chem. Chim. Ther., 14 <1979>, 411-414 discloses a process for producing 3,5-bis(trifluoromethyl)benzaldehyde by a Grignard reaction between 3,5-bis(trifluoromethyl)bromobenzene and triethoxyethane.

Nodiff, E. A. et al., J. Med. Chem., 15 <1972>, 775-780 discloses that 3,5-bis(trifluoromethyl)benzoic acid is reacted with thionyl chloride to obtain 3,5-bis(trifluoromethyl)benzoyl chloride, followed by reduction with Li<AlH(OtBu)₃> in diglyme.

Stogryn, E. L., J. Med. Chem., 16 <1973>, 1399-1401 or Porwisiak, Jacek; Schlosser, Manfred, Chem. Ber., 129 <1996> 2, 233-236 discloses a process in which 3,5-bis(trifluoromethyl)bromobenzene is subjected to lithionation by BuLi, followed by reaction with DMF.

Lockhart, Joyce C.; McDonnell, Martin B.; Clegg, William; Hill, M. N. Stuart, J. Chem. Soc. Perkin Trans. 2, <1987>, 639-650 discloses a reduction of 3,5-bis(trifluoromethyl)benzonitrile by tin chloride and hydrogen chloride.

Blackburn, Christopher; Childs, Ronald F.; Cremer, Dieter; Gauss, Juergen, J. Amer. Chem. Soc., 107 <1985> 8, 2442-2448 discloses an oxidation of 3,5-bis(trifluoromethyl)benzyl alcohol by chromic acid.

Patent: I. G. Farbenind., D. R. P. 670833 1935., Friedlaender, 25 118 discloses a reaction of 2-difluoromethyl-1,4-bis(trifluoromethyl)benzene with sulfuric acid.

The above-mentioned conventional processes are not satisfactory in industrially producing 3,5-bis(trifluoromethyl)benzaldehyde in a large amount. Thus, there has been a demand for a process for easily producing 3,5-bis(trifluoromethyl)benzaldehyde, which is useful as an intermediate for medicines, in an industrial scale.

### SUMMARY OF THE INVENTION

It is therefore an object of the present invention to provide a process for easily and efficiently producing bis(trifluoromethyl)benzaldehyde in an industrial scale.

According to the present invention, there is provided process for producing a bis(trifluoromethyl)benzaldehyde represented by the general formula [1].

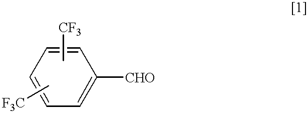

This process comprises reacting a mono-substituted bis(trifluoromethyl)benzene, represented by the general formula [2], with carbon monoxide and hydrogen in the presence of a catalyst and a base, said catalyst comprising a palladium compound and a phosphine,

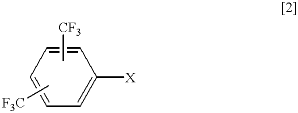

wherein X is a halogen atom selected from F, Cl, Br and I, a trifluoromethanesulfonate group, or a pentafluoroethanesulfonate group.

### DESCRIPTION OF THE PREFERRED EMBODIMENTS

It is possible to very easily and efficiently produce bis(trifluoromethyl)benzaldehyde represented by the general formula (1), which is useful as an intermediate for medicines, agricultural chemicals and various functional materials, in an industrial scale by the above-mentioned process of the invention.

Concrete examples of the mono-substituted bis(trifluoromethyl)benzene represented by the general formula [2] are 2,3-bis(trifluoromethyl)fluorobenzene, 2,3-bis(trifluoromethyl)chlorobenzene, 2,3-bis(trifluoromethyl)bromobenzene, 2,3-bis(trifluoromethyl)iodobenzene, 2,3-bis(trifluoromethyl)phenyl trifluoromethanesulfonate, 2,3-bis(trifluoromethyl)phenyl pentafluoroethanesulfonate, 2,4-bis(trifluoromethyl)fluorobenzene, 2,4-bis(trifluoromethyl)chlorobenzene, 2,4-bis(trifluoromethyl)bromobenzene, 2,4-bis(trifluoromethyl)bodobenzene, 2,4-bis(trifluoromethyl)phenyl trifluoromethanesulfonate, 2,4-bis(trifluoromethyl)phenyl pentafluoroethanesulfonate, 2,5-bis(trifluoromethyl)fluorobenzene, 2,5-bis(trifluoromethyl)fluorobenzene, 2,5-bis(trifluoromethyl)chlorobenzene, 2,5-bis(trifluoromethyl)bromobenzene, 2,5-bis(trifluoromethyl)iodobenzene, 2,5-bis(trifluoromethyl)phenyl trifluoromethanesulfonate, 2,5-bis(trifluoromethyl)phenyl pentafluoroethanesulfonate, 2,6-bis(trifluoromethyl)fluorobenzene, 2,6-bis(trifluoromethyl)chlorobenzene, 2,6-bis(trifluoromethyl)bromobenzene, 2,6-bis(trifluoromethyl)iodobenzene, 2,6-bis(trifluoromethyl)phenyl trifluoromethanesulfonate, 2,6-bis(trifluoromethyl)phenyl pentafluoroethanesulfonate, 3,4-bis(trifluoromethyl)fluorobenzene, 3,4-bis(trifluoromethyl)chlorobenzene, 3,4-bis(trifluoromethyl)bromobenzene, 3,4-bis(trifluoromethyl)iodobenzene, 3,4-bis(trifluoromethyl)phenyl trifluoromethanesulfonate, 3,4-bis(trifluoromethyl)phenyl pentafluoroethanesulfonate, 3,5bis(trifluoromethyl)fluorobenzene, 3,5-bis(trifluoromethyl)chlorobenzene, 3,5-bis(trifluoromethyl)bromobenzene, 3,5-bis(trifluoromethyl)iodobenzene, 3,5-bis(trifluoromethyl)phenyl trifluoromethanesulfonate, and 3,5-bis(trifluoromethyl)phenyl pentafluoroethanesulfonate.

Examples of the palladium compound used in the invention are palladium acetate and palladium chloride.

It is possible to define the phosphine used in the invention as a compound prepared by replacing hydrogen atom(s) of PH₃ with the same or different alkyl groups or aryl groups. Concrete examples of the phosphine are triphenylphosphine, triethylphosphine, trimethylphosphine, methyldiphenylphosphine, dimethylphenylphosphine, tri-o-tolylphosphine, tri-m-tolylphosphine, tri-p-tolylphosphine, tribenzylphosphine, dicyclohexylphenylphosphine, 1,2-bis(diphenylphosphino)ethane, 1,3-bis(diphenylphosphino)propane, 1,4-bis(diphenylphosphino)butane, and 1,1′-bis(diphenylphosphino)ferrocene.

Examples of the base used in the invention are tertiary amines (e.g., triethylamine, tripropylamine, triallylamine, N,N-dimethylaniline, N,N-diethylaniline, pyridine, and N-methylmorpholine), acetates (e.g., sodium acetate and potassium acetate), and inorganic bases (e.g., sodium hydroxide, potassium hydroxide, sodium carbonate and potassium carbonate).

The reaction of the invention may be conducted in a solvent or without using any solvent. It is, however, preferable to conduct the reaction in a solvent. Examples of the solvent are aliphatic hydrocarbons (e.g., pentane, hexane, heptane and octane), aromatic hydrocarbons (e.g., benzene, toluene and xylene), ethers (e.g., diethyl ether, dioxane, tetrahydrofuran (THF), and ethylene glycol dimethyl ether), ketones (e.g., acetone, methyl ethyl ketone, and methyl isobutyl ketone), nitriles (e.g., acetonitrile), tertiary amines (e.g., pyridine), acid amides (e.g., N,N-dimethylformamide (DMF) and N,N-dimethylacetoamide (DMAc)), and sulfur-containing compounds (e.g., dimethylsulfoxide (DMSO) and sulforane).

The amount of the palladium compound to be used in the process may be 0.00001-0.1 moles, preferably 0.00005-0.07 moles, more preferably 0.0001-0.05 moles, per mole of the mono-substituted bis(trifluoromethyl)benzene. If it is less than 0.00001 moles, the reaction may not proceed sufficiently, thereby lowering yield. This is economically disadvantageous. Furthermore, the reaction rate may become too low, thereby making the period of time for completing the reaction too long. Even if it is greater than 0.1 moles, the reaction rate and the yield may not improve further. Thus, this is economically disadvantageous.

The amount of the phosphine to be used in the process may be 0.00002-0.3 moles, preferably 0.0001-0.2 moles, more preferably 0.0002-0.1 moles, per mole of the mono-substituted bis(trifluoromethyl)benzene. If it is less than 0.00002 moles, the reaction may not proceed sufficiently, thereby lowering yield. This is economically disadvantageous. Furthermore, the reaction rate may become too low, thereby making the period of time for completing the reaction too long. Even if it is greater than 0.3 moles, the reaction rate and the yield may not improve further. Thus, this is economically disadvantageous.

The amount of the base to be used in the process may be 1.0-10.0 moles, preferably 1.0-5.0 moles, more preferably 1.0-2.0 moles, per mole of the mono-substituted bis(trifluoromethyl)benzene. If it is less than 1.0 mole, the reaction may not proceed sufficiently, thereby lowering yield. Furthermore, it may become necessary to conduct a post-treatment for removing or collecting the unreacted mono-substituted bis(trifluoromethyl)benzene. Even if it is greater than 10.0 moles, the yield of the target bis(trifluoromethyl)benzaldehyde may not change significantly. Furthermore, an excess of the base may remain unreacted in a large amount. This is economically disadvantageous. Furthermore, it may become necessary to conduct a post-treatment for removing the unreacted base.

The amount of hydrogen to be used in the process may be 0.1-3.0 moles, preferably 0.2-2.0 moles, more preferably 0.5-1.5 moles, per mole of carbon monoxide.

The process of the invention can be conducted, as follows. At first, a reaction vessel is charged with the mono-substituted bis(trifluoromethyl)benzene, a palladium compound, a phosphine, a base, and if necessary a solvent. After that, the reaction vessel is stopped, and its atmosphere is replaced with a gas mixture of carbon monoxide and hydrogen, thereby proceeding the reaction under normal (atmospheric) pressure or a pressurized condition. The pressure of this gas mixture may be from normal pressure to 20 MPa, preferably 0.2-15 MPa, more preferably 0.5-10 MPa. If it is lower than normal pressure, the reaction may not proceed sufficiently, thereby lowering yield. This is economically disadvantageous. Furthermore, the reaction rate may become too low, thereby making the period of time for completing the reaction too long. Even if it is higher than 20 MPa, the reaction rate and the yield of the target bis(trifluoromethyl)benzaldehyde may not improve further. Furthermore, it may become necessary to use a special pressure-proof reaction vessel.

The reaction temperature of the process may be from 30° C. to 200° C., preferably 50-180° C., more preferably 60-150° C. If it is lower than 30° C., the reaction may not proceed sufficiently, thereby lowering yield. This is economically disadvantageous. Furthermore, the reaction rate may become too low, thereby making the period of time for completing the reaction too long. If it is higher than 200° C., the reactants may be decomposed during the reaction, thereby lowering yield. This is economically disadvantageous. Furthermore it may become necessary to conduct a post-treatment for removing a decomposition product(s).

It is possible to obtain a crude product by conducting a normal post-treatment(s) after the reaction. The obtained crude product can be subjected to a purification (e.g., solvent extraction, distillation, recrystallization, and column chromatography), thereby obtaining the target bis(trifluoromethyl)benzaldehyde (e.g., 3,5-bis(trifluoromethyl)benzaldehyde).

Examples of the target bis(trifluoromethyl)benzaldehyde, which can be produced by the process, are 2,3-bis(trifluoromethyl)benzaldehyde, 2,4-bis(trifluoromethyl)benzaldehyde, 2,5-bis(trifluoromethyl)benzaldehyde, 2,6-bis(trifluoromethyl)benzaldehyde, 3,4-bis(trifluoromethyl)benzaldehyde, and 3,5-bis(trifluoromethyl)benzaldehyde.

The following nonlimitative examples are illustrative of the present invention.

### EXAMPLE 1

Production of 3,5-bis(trifluoromethyl)benzaldehyde

A 300-ml stainless steel autoclave was charged with 70.0 g (0.206 mol) of 3,5-bis(trifluoromethyl)iodobenzene, 23.0 g (0.227 mol) of triethylamine, 140 g of DMF, 0.46 g (2.05 mmol) of palladium acetate, and 2.50 g of tri-o-tolylphosphine. Then, the atmosphere of the autoclave was replaced two times with a gas mixture of carbon monoxide and hydrogen (CO:H₂=1:1 by volume). After that, the reaction was conducted for 5 hr at 125° C. under a pressure of the gas mixture of 1.5 MPa. After the reaction, the autoclave was cooled down to room temperature, and the gas was purged. Then, the reaction mixture was washed with 200 ml of water. The precipitated solid matter was removed by filtration. The resulting organic layer was dried with magnesium sulfate anhydride. Then, magnesium sulfate was separated by filtration. The resulting filtrate (crude product) was found by a gas chromatography (detector: FID; column: DB-1; column size: 0.25 mm×30 m) to contain 9.75% of 1,3-bis(trifluoromethyl)benzene, 71.0% of 3,5-bis(trifluoromethyl)benzaldehyde, and 19.25% of others. The crude product was distilled under vacuum, thereby obtaining 21.0 g of 3,5-bis(trifluoromethyl)benzaldehyde as a fraction of 64-68° C./10 mmHg. This target product was found by the gas chromatography to have a purity of 99.0%.

### EXAMPLES 2-10

Production of 3,5-bis(trifluoromethyl)benzaldehyde

In each of these examples, Example 1 was repeated except in that the type of the starting material (i.e., the mono-substituted bis(trifluoromethyl)benzene), the type of phosphine, the type of solvent and the reaction temperature were changed as shown in Table 1 and that the distillation of the crude product was omitted. The results of Examples 2-10 are shown in Table 2.

TABLE 1

| | | Pd | | | | |
|---|---|---|---|---|---|---|
| | Starting | Com- | | | Temp. | Pressure |
| | Material | pound | Phosphine | Solvent | (° C.) | (MPa) |
| Ex. 2 | MBT-I* | Pd(OAc)₂ | Ph₃P* | DME* | 120 | 1.5 |
| Ex. 3 | MBT-I* | Pd(OAc)₂ | Ph₃P* | CH₃CN* | 120 | 1.5 |
| Ex. 4 | MBT-I* | Pd(OAc)₂ | dppp* | DMF* | 130 | 1.5 |
| Ex. 5 | MBT-I* | Pd(OAc)₂ | dppp* | DME* | 130 | 1.5 |
| Ex. 6 | MBT-I* | Pd(OAc)₂ | dhpp* | CH₃CN* | 130 | 1.5 |
| Ex. 7 | MBT-I* | Pd(OAc)₂ | dhpp* | toluene | 130 | 1.5 |
| Ex. 8 | MBT-I* | Pd(OAc)₂ | Tol₃P* | CH₃CN* | 130 | 1.5 |
| Ex. 9 | MBT-I* | Pd(OAc)₂ | dppf* | DMF* | 130 | 1.5 |
| Ex. 10 | MBT- | Pd(OAc)₂ | Tol₃P* | DMF* | 150 | 1.5 |
| | Br* | | | | | |

*MBT-I: 3,5-bis(trifluoromethyl)iodobenzene;
MBT-Br: 3,5-bis(trifluoromethyl)bromobenzene;
Ph₃P: triphenylphosphine;
dppp: 1,3-bis(diphenylphosphino)propane;
dhpp: dicyclohexylphenylphosphine;
Tol₃P: tri-o-tolylphosphine;
dppf: 1,1′-bis(diphenylphosphino)ferrocene;
DME: ethylene glycol dimethyl ether;
CH₃CN: acetonitrile; and
DMF: N,N-dimethylformamide.

TABLE 2

| | Reaction Products (%) | | | |
|---|---|---|---|---|
| | | Target | Unreacted | |
| | By-product | Product | Starting | |
| | (BTMB*) | (BMBA*) | Material | Others |
| Ex. 2 | 25.9 | 15.3 | 0.2 | 58.6 |
| Ex. 3 | 3.0 | 8.2 | 77.1 | 11.8 |
| Ex. 4 | 23.8 | 55.2 | 6.2 | 14.8 |
| Ex. 5 | 47.4 | 25.9 | 24.3 | 2.4 |
| Ex. 6 | 18.1 | 4.9 | 71.1 | 5.9 |
| Ex. 7 | 75.6 | 9.4 | — | 15.0 |
| Ex. 8 | 15.4 | 52.7 | 0.4 | 31.6 |
| Ex. 9 | 17.0 | 60.9 | — | 22.1 |
| Ex. 10 | 30.2 | 0.2 | 64.8 | 4.8 |

*BTMB: 1,3-bis(trifluoromethyl)benzene; and
BMBA: 3,5-bis(trifluoromethyl)benzaldehyde.

The entire disclosure of Japanese Patent Application No. 2000-193314 filed on Jun. 27, 2000, including specification, claims and summary, of which priority is claimed in the present application, is incorporated herein by reference in its entirety.

What is claimed is:

1. A process for producing a bis(trifluoromethyl)benzaldehyde represented by the general formula [1], said process comprising reacting a mono-substituted bis(trifluoromethyl)benzene, represented by the general formula [2], with carbon monoxide and hydrogen in the presence of a catalyst and a base, said catalyst comprising a palladium compound and a phosphine, 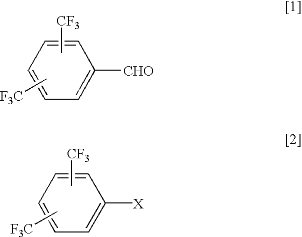
wherein X is a halogen atom selected from F, Cl, Br and I, a trifluoromethanesulfonate group, or a pentafluoroethanesulfonate group.

2. A process according to claim 1, wherein said mono-substituted bis(trifluoromethyl)benzene is selected from the group consisting of 3,5-bis(trifluoromethyl)halogenobenzene, 3,5-bis(trifluoromethyl)phenyl trifluoromethanesulfonate and 3,5-bis(trifluoromethyl)phenyl pentafluoroethanesulfonate, and
wherein said bis(trifluoromethyl)benzaldehyde is 3,5-bis(trifluoromethyl)benzaldehyde.

3. A process according to claim 1, wherein said mono-substituted bis(trifluoromethyl)benzene is 3,5-bis(trifluoromethyl)halogenobenzene and
wherein said bis(trifluoromethyl)benzaldehyde is 3,5-bis(trifluoromethyl)benzaldehyde.

4. A process according to claim 1, wherein said X is a bromine or iodine atom.

5. A process according to claim 1, wherein said palladium compound is palladium acetate or palladium chloride.

6. A process according to claim 1, wherein said phosphine is selected from the group consisting of triphenylphosphine, tri-o-tolylphosphine, dicyclohexylphenylphosphine, 1,3-bis(diphenylphosphino)propane and 1,1′-bis(diphenylphosphino)ferrocene.

7. A process according to claim 1, wherein said base is triethylamine.

8. A process according to claim 1, wherein said reacting is conducted in a solvent.

9. A process according to claim 8, wherein said solvent is selected from the group consisting of ethylene glycol dimethyl ether, acetonitrile, N,N-dimethylformamide, and toluene.

10. A process according to claim 1, wherein said palladium compound is in an amount of 0.00001-0.1 moles per mole of said mono-substituted bis(trifluoromethyl)benzene.

11. A process according to claim 1, wherein said phosphine is in an amount of 0.00002-0.3 moles per mole of said mono-substituted bis(trifluoromethyl)benzene.

12. A process according to claim 1, wherein said base is in an amount of 1.0-10.0 moles per mole of said mono-substituted bis(trifluoromethyl)benzene.

13. A process according to claim 1, wherein said hydrogen is in an amount of 0.1-3.0 moles per mole of said carbon monoxide.

14. A process according to claim 1, wherein said reacting is conducted under atmospheric pressure or a pressure of 20 MPa or lower.

15. A process according to claim 1, wherein said reacting is conducted at a temperature of 30-200° C.

16. A process according to claim 15, wherein said reacting is conducted at a temperature of 120-150° C.
